(19) United States
(12) Patent Application Publication
(10) Pub. No.: US 2026/0085038 A1
(43) Pub. Date: Mar. 26, 2026

(54) PROCESS FOR OBTAINING MIXTURES CONTAINING METHIONINE AND POTASSIUM HYDROGENCARBONATE

(71) Applicant: Evonik Operations GmbH, Essen (DE)

(72) Inventors: Karl-Georg FEY, Neuberg (DE); Claudia DYBALLA, München (DE)

(73) Assignee: Evonik Operations GmbH, Essen (DE)

(21) Appl. No.: 18/870,513

(22) PCT Filed: Jun. 7, 2023
(86) PCT No.: PCT/EP2023/065244

§ 371 Date: Nov. 29, 2024

(30) Foreign Application Priority Data
Jun. 17, 2022 (EP) 22179609.7

(51) Int. Cl. C07C 319/20; C07C 319/28

(52) U.S. Cl. CPC C07C 319/20; C07C 319/28

(57) ABSTRACT

A process for obtaining mixtures including methionine and potassium hydrogencarbonate from aqueous solutions or suspensions including titratable potassium in the form of potassium hydrogencarbonate and/or potassium carbonate, methionine and 4.5-12.0% by weight of methionyl-methionine, including supplying the employed solutions or suspensions with CO₂ (carbonating) at a temperature of 15 to 60° C. to precipitate a mixture including methionine and potassium hydrogencarbonate as precipitate which includes on average not more than 6.5% by weight of met-met and separating said precipitate from the mother liquor.

### FIELD OF THE INVENTION

The present invention relates to a process for obtaining mixtures containing methionine and potassium hydrogencarbonate and optionally potassium carbonate from aqueous solutions or suspensions containing methionine, potassium carbonate, potassium hydrogencarbonate and methionyl-methionine and in particular a method for recovery of methionine and potassium hydrogencarbonate from methionine mother liquors enriched with met-met.

### BACKGROUND OF THE INVENTION

The amino acid methionine is employed in many fields, for example pharmaceutical, health and fitness products, but particularly as a feedstuff additive in many feedstuffs for various livestock. On an industrial scale, methionine is produced chemically via the Bucherer-Bergs reaction, which is a variant of the Strecker synthesis. The starting substances 3-methylmercaptopropanal (NMP, produced from 2-propenal and methylmercaptan), hydrocyanic acid (hydrogen cyanide), ammonia and carbon dioxide are reacted to afford 5-(2-methylmercaptoethyl)hydantoin (methionine hydantoin) and this is subsequently subjected to alkaline hydrolysis with alkali metal hydroxide and/or alkali metal carbonate and alkali metal hydrogencarbonate, for example potassium hydroxide and/or potassium carbonate and potassium hydrogen carbonate, to afford alkali metal methionate (for example potassium methioninate) (see formula 1). Methionine is finally liberated from its alkali metal salt by acidification, for example with carbon dioxide (carbonation) (see formula 2), and is filtered off as a precipitate from the mother liquor containing alkali metal carbonate and alkali metal hydrogen carbonate (for example potassium carbonate and potassium hydrogen carbonate). The filtrate, the mother liquor containing alkali metal hydrogencarbonate (for example potassium carbonate and potassium hydrogencarbonate) (1st mother liquor) is recirculated based on the Degussa potassium recycle process for saponification of methionine hydantoin (see for example EP 780370 A2).

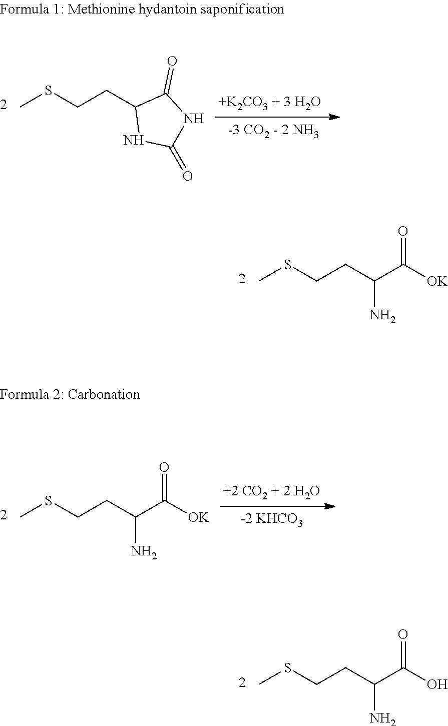

As in all recycle processes, this process too requires discharging of the mother liquor to prevent the byproducts formed exceeding the tolerable amount. However, the discharged mother liquor still contains the product methionine, alkali metal carbonate and alkali metal hydrogencarbonate (for example potassium carbonate and potassium hydrogencarbonate) which is useful for the saponification of methionine hydantoin. To recover as much methionine and potassium hydrogencarbonate from the mother liquor as possible the mother liquor to be discharged may be subjected to a second carbonation.

There are various processes for obtaining methionine and potassium hydrogen carbonate from the 1st mother liquor via a second carbonation, as reported for example in DE2421167A1 and EP839804A2. The recited EP specification requires a water-soluble solvent for precipitation of the methionine which entails additional complexity and possible impurities.

A further obtaining of methionine and potassium hydrogencarbonate from mother liquors, for example from the mother liquor of the second carbonation (2nd mother liquor) is more difficult. This is because impurities are markedly more enriched in such mother liquors and these impurities can disrupt subsequent process steps. Thus, EP1760074A1 discloses a method for recovery of methionine and potassium hydrogencarbonate from the mother liquor of the 2nd carbonation via a 3rd carbonation. The resulting precipitated mixture of methionine and potassium hydrogencarbonate is separated by filtration and may be directly recycled to the hydantoin saponification. However, in this method the precipitate of the 3rd carbonation is not readily filterable, as reported in EP2133328A2 and in EP2186798A1. The reason for this is that the mother liquor of the second carbonation has become markedly enriched in the byproduct methionyl-methionine (met-met) and met-met adversely affects the filterability of the precipitate of the 3rd carbonation. It is therefore sought to reduce the content of met-met in the 2nd mother liquor. Accordingly, in EP2133328A2 and in EP2186798A1, the mother liquor of the 2nd carbonation is initially concentrated and subsequently heated to an elevated temperature, for example to 180° C. As a result of this treatment met-met in the 2nd mother liquor is partially cleaved into methionine. The thus-obtained mother liquor is then suitable for a recovery of the methionine and potassium hydrogencarbonate present therein by a third carbonation, as described for example in EP2133328A/EP2186798A1 or by recycling of the mother liquor to the 2nd carbonation as described in EP2186797A1.

An alternative method for reducing the content of met-met is disclosed in EP2133329A2. According to EP2133329A the methionine hydantoin saponification forms less met-met when the hydrolysis is performed in an unstirred reactor (no backmixing) and after removal of CO₂ and water the remaining hydrolysis solution is further heated in a separate reactor. This accordingly requires two separate reactors and also fails to avoid heating of the basic solution at elevated temperature which is likewise disadvantageous. The use of the auxiliary polyvinyl alcohol, which entails additional complexity and cost and results in corresponding residues in the product, is also disadvantageous.

It is also known from EP1840119A2 that the conditions required above for a met-met cleavage in the basic methionine mother liquor are very corrosive (high concentration and temperature) and accordingly costly materials are required for the reactors (for example zirconium or super duplex steel). Furthermore, the abovementioned conditions for the cleavage of met-met to methionine are very harsh. These conditions may likewise result in the decomposition of methionine which is also disadvantageous, as is the increased energy demand for this additional process step.

There is therefore a need for novel methods for recovering methionine and potassium hydrogencarbonate from methionine from met-met-enriched mother liquors, for example the mother liquor from the 2nd carbonation, which exhibit the disadvantages of the methods described in the prior art only to a reduced extent, if at all.

The process for recovering value substances from the discharged mother liquor through carbonation and separation has a central object: The separation of the precipitate generated in the carbonation (this contains especially the value substances potassium hydrogencarbonate and methionine) and the mother liquor (this is rich in secondary components such as formate and met-met). The precipitate is recycled into the process and the remaining mother liquor (for example the 2nd or 3rd mother liquor) constitutes the waste product.

If this solid-liquid separation does not work well, i.e. when the precipitate is difficult to separate, difficult to filter and can therefore be recycled into the process only with a lot of mother liquor, this has the result that a lot of secondary components are also returned to the hydrolysis step—the hydantoin saponification—and these thus build up in the process, with the result that more 1st mother liquor must be discharged to remove these byproducts from the circuit again. The filterability of the precipitate thus has a decisive effect on the throughput through the entire workup of the discharged 1st mother liquor. The filterability is therefore decisive for the economy of the entire workup.

In the recovery of methionine and hydrogencarbonate from met-met-enriched methionine mother liquors, as arises from the abovementioned patent specifications, it is precisely the filtration that is problematic. According to the abovementioned specifications this is due to the higher concentration of met-met in the 2nd mother liquor. For this reason these also describe several processes for reducing the met-met content in the mother liquor to improve the filterability of the precipitate.

Thus the 2nd mother liquor is for example first heated for a duration of 0.3 to 10 hours (EP2186797A1) to high temperatures (for example 160-180° C.) to cleave met-met into methionine before the carbonation and filtration. This facilitates the filtration that follows.

However, the described solutions have the disadvantage that costly additional equipment is required (for example a corrosion-resistant reactor with sufficient residence time and a heat exchanger), additional energy input is required and also the value substance methionine undergoes partial decomposition. This re-forms secondary components.

### Problem

The problem addressed by the present invention was accordingly that of providing a process for recovering mixtures containing methionine, potassium hydrogencarbonate and optionally potassium carbonate from aqueous solutions or suspensions containing methionine, optionally potassium carbonate, potassium hydrogencarbonate and methionyl-methionine, in particular from mother liquors of methionine production appreciably enriched in methionyl-methionine, where methionine, potassium hydrogencarbonate and optionally potassium carbonate present is precipitated in the highest possible proportion and this is filterable as readily as possible, i.e. exhibits the best possible filterability.

A further problem directly associated therewith was that of providing an improved process for producing methionine where a small amount of waste products is generated/requires disposal.

### DESCRIPTION OF THE INVENTION

The present process solves the problem of poor filterability of the precipitate generated in the carbonation of met-met-enriched mother liquors in a manner distinct from the prior art. According to the invention the precipitate from the carbonation contains the substances methionine, potassium hydrogencarbonate and optionally relatively small proportions of potassium carbonate and the accompanying mother liquor also contains appreciable amounts of methionyl-methionine. The finding upon which the invention is based is that the enriched met-met does not appreciably disrupt the filtration provided the precipitated content of methionyl-methionine, i.e. that present in the precipitate, does not exceed a critical value, i.e. remains largely dissolved in the mother liquor. The filtration becomes difficult only when met-met precipitates in appreciable quantities during the carbonation and is thus present in the precipitate as crystals.

The abovementioned object is accordingly achieved by providing a process for obtaining mixtures containing methionine, potassium hydrogencarbonate and optionally potassium carbonate from aqueous solutions or suspensions containing
preferably 6.0-18.0% by weight of titratable potassium in the form of potassium hydrogencarbonate and/or potassium carbonate,
preferably 2.5-8.0% by weight of methionine and 4.50-12.0% by weight of methionyl-methionine, characterized in that it comprises supplying the employed solutions or suspensions with CO₂ (carbonating) at a temperature of 15-60° C. to precipitate a mixture containing methionine, potassium hydrogencarbonate and optionally potassium carbonate as precipitate which contains on average not more than 6.5% by weight, preferably not more than 0.01% to 5.0% by weight, in particular not more than 0.01% to 3.0% by weight of met-met. The desired met-met content may be determined on a continuous basis using samples from the separated precipitate by HPLC for example.

This achieves particularly good filterabilities and thus overcomes the abovementioned disadvantages of the processes from the prior art. The employed aqueous solutions or suspensions have an alkaline pH of about 11 to 12 which is reduced by the carbonation, preferably to a pH of 7.8 to 9.5, particularly preferably of 8.3 to 9.5, in particular of 8.4 to 9.5, and very particularly preferably of 8.4 to 9.0, measured with a glass electrode at the temperature established in each case. The met-met content in the precipitate is markedly dependent on the pH in the carbonated mother liquor and this can therefore also be used to control the met-met content in the precipitate.

The separation of the precipitate may be carried out for example by filtration or centrifugation, in particular using corresponding apparatuses also employable on an industrial scale and known to those skilled in the art which are preferably employed on a continuous or semi-continuous basis so the industrially customary product throughputs can be run. To control and regulate the pH and thus also indirectly the technical effect of good filterability it is possible either to take regular samples and measure these with a glass electrode (cf. examples) or to choose continuous pH measurement to allow very easy adjustment of the desired pH and thus filterability. For direct control and regulation of the met-met content and thus also of the technical effect of good filterability it is possible to take regular samples from the mother liquor and measure them by HPLC (cf. examples). However, HPLC determination of the met-met content advantageous according to the invention can also proceed in automated fashion.

Preference is given to a process characterized in that it comprises bringing the employed solutions or suspensions, optionally by concentration, to a content of
6.0-18% by weight of titratable potassium in the form of potassium carbonate and/or potassium hydrogencarbonate,
2.5-8.0% by weight of methionine and
4.5-12.0% by weight of methionyl-methionine and then
supplying CO₂ (carbonation) at a temperature of 15-60° C., preferably 25-55° C., until a pH of 7.8 to 9.5, preferably 8.3 to 9.5, particularly preferably of 8.4 to 9.5, in particular of 8.4 to 9.0 is achieved, measured with a glass electrode at the established temperature, to precipitate a mixture containing methionine, potassium hydrogencarbonate and optionally potassium carbonate as precipitate and separating said precipitate from the mother liquor, where the precipitate contains on average not more than 6.5% by weight, preferably not more than 0.01% to 5.0% by weight, in particular not more than 0.01% to 3.0% by weight of met-met.

The present invention accordingly provides a process which makes it possible to achieve the recovery of methionine and potassium hydrogencarbonate from met-met-enriched methionine mother liquors by selecting the process parameters (in particular the carbonation) such that during the carbonation the greatest possible amount of methionine and potassium hydrogencarbonate undergo precipitation but simultaneously the met-met in the precipitated precipitate does not exceed a critical value, i.e. very largely remains dissolved in the mother liquor.

In the present invention a cleavage of met-met before the carbonation is no longer necessary, thus making it possible to achieve considerable energy and cost savings. The process according to the invention in its abovementioned variants can even achieve an improvement in the filterability of the precipitate from the carbonation. This is apparent for example from the markedly lower filtration resistance of 1.2*10⁶ m/kg in inventive example 1 compared to the value described in EP2186797A1 example 1 (2nd crystallization) of 1.6*10⁹ m/kg or compared to the value described in EP2133328A2 example 1 (3rd crystallization) of 0.64*10¹⁰ m/kg.

According to the invention it is in particular the 2nd mother liquor of the 2nd carbonation that is concentrated. A low pressure is selected (for example 300 mbara) to effect a gentle concentration of the mother liquor at low-temperature. This avoids cleavage of methionine and met-met. The mother liquor is for example concentrated by a factor of 2. The concentration of free potassium is then 13.4% by weight for example and the concentration of methionine is 4.9% by weight for example. Met-met too is then enriched; the concentration thereof is 7% by weight for example. (See example 1). This solution is then carbonated by addition of CO₂. The pressure is in the range 1-6, preferably 1.5-2.5 bara to reduce foaming of the suspension during its decompression. The temperature is between 15-60° C., preferably 25-35° C., to avoid the use of cold water and instead allow the use of energetically more favorable cooling water. The residence time in the CO₂ supplying is between 20 and 180 minutes, preferably about 60 minutes. In the reactor the pH of the suspension is measured at the respective temperature with a glass electrode. This is adjusted via the CO₂ addition. The concentrated mother liquor has a pH of about 11. The mother liquor in the reactor is not completely carbonated (i.e. to equilibrium at the respective pressure) at this point. By contrast, only sufficient CO₂ to achieve a pH of 7.8 to 9.5, for example 8.5, is added.

The inventors have found that the solubility of met-met in this matrix is particularly dependent on temperature and pH. The target pH in the reactor (and thus the CO₂ amount) is advantageously chosen such that only a small proportion of met-met, namely not more than 6.5% by weight, preferably not more than 0.1% to 5.0% by weight, particularly preferably not more than 0.1% to 3.0% by weight, is present in the precipitate. The met-met content is typically determined by HPLC on initially filtration-moist and subsequently acetone-washed and dried samples of the precipitate. This is naturally a cross-sectional value of the crystalline met-met proportion in the precipitate and the met-met proportion in the mother liquor adhering to the precipitate. However, this sum value/average value is also indirectly a measure of the relevant crystalline proportion of met-met, and so may be used as a manipulated variable to adjust the filterability.

The preferred target pH is therefore dependent on:
concentration of met-met in the filtrate of the 2nd carbonation
concentration of potassium, methionine and met-met after the concentration which in each case correlates with the concentration factor which is typically 1.5 to 2.5
temperature in the reactor

After the carbonation the precipitate is typically filtered. Since the mother liquor has not been carbonated to equilibrium the precipitate contains only relatively small amounts of met-met in addition to methionine and potassium hydrogencarbonate. The precipitate is recycled to the process, preferably upstream of the hydantoin saponification (reaction step comprising the hydrolysis of 5-[2-(methylthio)ethyl]imidazolidin-2,4-dione). Any met-met present therein is cleaved into methionine in the hydantoin saponification. Similarly to the recycling of the methionine present in the precipitate this increases the yield of the methionine process. As a result of the concentration the filtrate is about 2 times richer in secondary components and is preferably disposed of. Due to the concentration by a factor of about 2 the amount of the filtrate is also reduced by a factor of about 2. This markedly reduces the liquid waste stream of the process.

The process is advantageously operated such that the pressure is in the range 1-6, preferably 1.5-2.5 bara.

Typical residence times are between 20 and 180 minutes, preferably about 60 minutes.

As aqueous solutions or suspensions containing methionine, potassium carbonate, potassium hydrogencarbonate and methionyl-methionine in the process according to the invention it is preferable to employ a mother liquor from the isolation of methionine in the process for producing methionine via alkaline methionine hydantoin saponification, as is described in principle in EP 780370 A2 for example.

The mother liquor is preferably the mother liquor from the filtration after repeated carbonation, in particular the 3rd carbonation, because the advantage of recovering otherwise lost value substances is greatest here.

The present invention also provides a mixture containing methionine, potassium hydrogencarbonate and optionally potassium carbonate produced according to the above-described process and a mixture containing methionine, KHCO₃ and met-met, wherein the met-met content is not more than 6.5% by weight, as is producible by the process according to the invention.

The present invention further provides the use of such mixtures for producing methionine, in particular as a saponification agent and additional methionine source. This has the great advantage of enhancing process economy through resource conservation and increasing yields, thus having a significant impact at a typical plant output of about 100 000 tons of methionine per annum.

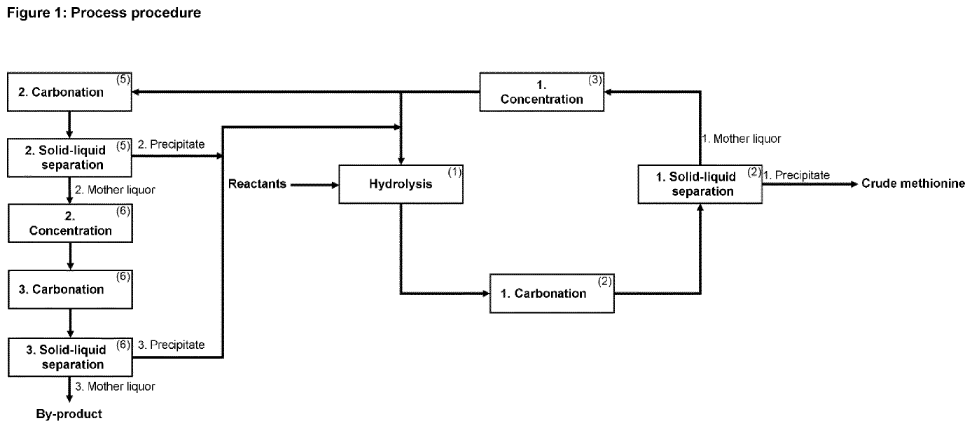

The invention further provides an overall process for producing methionine comprising the following steps (1) to (6) (see also FIG. 1, block diagram with principle process procedure):
(1) a reaction step comprising hydrolysis of 5-[2-(methylthio)ethyl]imidazolidin-2,4-dione in the presence of a basic potassium compound to afford a hydrolyzate containing potassium methioninate and potassium methionyl methioninate;
(2) a first crystallization step comprising introduction of carbon dioxide (1st carbonation) into the hydrolyzate obtained in step (1) to obtain a suspension containing methionine, methionyl-methionine, potassium hydrogencarbonate and optionally potassium carbonate and to precipitate methionine and separating the suspension into a methionine-containing 1st precipitate and a methionyl-methionine-containing 1st mother liquor;
(3) concentrating the 1st mother liquor obtained in step (2) (1st concentration step),
(4) recycling a first portion of the concentrated 1st mother liquor from (3) into reaction step (1)
(5) a second crystallization step which comprises introduction of carbon dioxide into the second portion of the concentrated 1st mother liquor from (3) (2nd carbonation) to precipitate methionine and potassium hydrogencarbonate and separating the resulting suspension into a 2nd precipitate and a 2nd mother liquor;
(6) a third crystallization step which comprises concentrating the 2nd mother liquor obtained in step (3), introducing carbon dioxide into the concentrated 2nd mother liquor to precipitate methionine and potassium hydrogencarbonate and separating the resulting suspension into a 3rd precipitate containing a mixture of methionine, potassium hydrogencarbonate and optionally potassium carbonate and a methionyl-methionine-containing 3rd mother liquor according to the above-described process, so that the 3rd precipitate contains not more than 6.5% by weight, preferably not more than 0.01% to 5% by weight, particularly preferably not more than 0.01% to 3% by weight, of met-met on average.

Preferably employed as the basic potassium compound is potassium hydroxide, potassium carbonate and/or potassium hydrogencarbonate and, at least supplementally, the abovementioned potassium hydrogencarbonate- and optionally potassium carbonate-containing precipitates from the carbonation steps.

By providing the overall process according to the invention it has been possible to solve the problem of providing an improved process for producing methionine where the yield of methionine is increased, more basic potassium is recycled from the mother liquor and thus a markedly smaller amount of waste products is generated/requires disposal. Against the backdrop of typical production output of about 100 000 tons per annum of a typical methionine plant, in particular having regard to the advantage of resource conservation, this is of high economic and ecological value.

Also preferred is a process where the 3rd precipitate is recycled into the hydrolysis step (1) or into the further steps (2) to (4) where on account of its high potassium content said precipitate serves to reduce the demand for replacement potassium and to recycle the methionine present.

It is also advantageous for the 1st mother liquor and the 3rd precipitate to be combined and subsequently recycled into the hydrolysis step (1) or the concentration step (3), which has the advantage that it reduces the complexity of the process.

The 3rd mother liquor obtained as waste in the process may be either simply disposed of or else supplied to a further isolation of value substances. These are especially the recirculated potassium hydrogencarbonate and the end product of the process, methionine.

### Examples

### Methods Used

### 1. High-Performance Liquid Chromatography (HPLC)

Chromatographic investigations (methionine and met-met) were performed using an HPLC instrument from Jasco on a suitable RP column with subsequent UV detection at 210 nm. The mobile phase was an acetonitrile-water mixture acidified with phosphoric acid. 10 μl of the respective sample solution was injected at a flow rate of 1 ml/min. The system was calibrated beforehand by injecting suitable calibration solutions of appropriate reference compounds from the methionine/met-met process, with evaluation by peak area comparison using the external standard method. The procedure of the standard method is known to those skilled in the art.

Sampling for determining met-met in the precipitate:

One part by volume of the filtered precipitate from the carbonation was collected on a laboratory filter funnel and washed with two-volume equivalents of acetone and suctioned off under reduced pressure in a waterjet vacuum. The remaining proportion of the mother liquor still adhering to precipitate which was slightly soluble in acetone was largely removed. Suction was applied for a further 5 min and the precipitate was then dried to a constant weight in a drying cabinet, thus removing the remaining acetone. Subsequently a suitable proportion of the thus-pretreated precipitate was weighed, dissolved in the HPLC eluent and diluted with further HPLC eluent to a suitable concentration and the thus-obtained sample solution injected into the HPLC instrument as elucidated above.

### 2. Potassium Titration

Alkaline potassium salts, such as potassium carbonate, potassium hydrogencarbonate and potassium methioninate are present in almost all process solutions of the methionine process and are here encompassed by the term “titratable potassium”. A titrimetric determination of “titratable potassium” aqueous solution was carried out, in particular in process solutions, as a collective parameter. This is carried out as a potentiometric titration with 0.1 molar HCl against the relevant potassium-containing alkaline aqueous process solutions up to pH 4.6. In the acid/base titration the acid consumption is reported as “titratable potassium”.

1 ml of 0.1 molar HCl corresponds to a weight equivalent of 3.91 mg of K⁺ for example according to the reaction with KHCO₃:

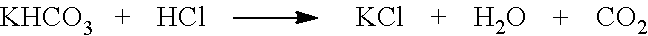 [SMILES: "KHCO3" ; Cl ; "KCl" ; O ; "CO2"]

Note: When the term equivalent (eq) is used this is to be understood as meaning mole equivalents (also moleq).

### 3. Measurement of Cake Resistance as a Measure for the Filterability of a Suspension

The so-called cake resistance was determined according to VDI Guideline VDI 2762 sheet 2, December 2010. Cake resistance is reported in 1/m². The area-based cake resistance α_{H} is reported in 1/m² and the mass-based cake resistance am is reported in m/kg. The two parameters are converted via the density of the dry filtercake (ρ₅ in kg/m³): α_{H}=α_{M}*ρₛ. The higher the cake resistance value the poorer the filterability. The filterability was also determined empirically by observation and comparison of different filtration tests. The following filterability ratings were awarded:
very good—good—acceptable—poor—very poor.

Inventive examples were accordingly given the ratings “very good”, “good” or “acceptable”.

### 4. Production of Methionine Hydantoin Saponification Solution Substantially According to EP 780370 A2 as a Starting Solution for the Examples

Initially, substantially according to EP 780370 A2, example 1 (page 10), a methionine hydantoin solution was produced from MMP, HCN and ammonia and CO₂ in the form of ammonium carbonate solution and therefrom, through saponification using an aqueous, titratable potassium, in particular in the form of K₂CO₃-containing solution substantially according to example 6 of EP 780370 A2, methionine hydantoin saponification solution (hydrolyzate) was produced. This hydrolysate, an alkaline solution containing in particular potassium methioninate and potassium methionyl methioninate, was largely neutralized using CO₂ (1st carbonation) analogously to example 7 of EP 780370 A2. The thus-precipitated 1st precipitate containing especially methionine was filtered off from the accompanying 1st mother liquor (1st moli) as described above.

The 1st moli was carbonated again (2nd carbonation) and the resulting precipitate filtered from the remaining 2nd moli. The resulting 2nd moli was in examples 1 to 12 subjected to further processing as starting solution 1, 2 and 3 with the composition in each case reported in Tab.1. This respective starting solution (2nd moli) was concentrated to a factor of 1.6 to 1.9 and the thus obtained concentrated 2nd moli was in each case sent to a 3rd carbonation. The thus-formed precipitate was in each case filtered off via a filtration unit and analyzed for titratable potassium K+, methionine and met-met. The filterability was assessed and in some cases also cake resistance determined (cf. table 1).

Table 1 provides an overview of the examples with the results of the carbonations and filtrations to elucidate the dependence of the filtration on the met-met concentration in the filtercake.

TABLE 1
Overview of adjusted parameters and results of examples 1 to 12

| | | Concentration in % | | | | | | Cake |
|---|---|---|---|---|---|---|---|---|
| | | by weight | | | | | | resistance |
| | | | | Met- | Carbonation | Carbonation | | α_{M} in |
| Example | Process step | K⁺ | Met | Met | T in ° C. | pH | Filterability | m/kg |
| | 2. Moli (starting | 7.0 | 2.6 | 3.7 | | | | |
| | solution 1 for | | | | | | | |
| | examples 1 and 2) | | | | | | | |
| | 2. Moli after | 13.4 | 4.9 | 7 | | | | |
| | concentration | | | | | | | |
| | by factor of 1.9 | | | | | | | |
| 1 | 3. Moli after | 8.0 | 3.5 | 7.8 | 30 | 8.7 | | |
| | carbonation | | | | | | | |
| | 3. Precipitate and | 26.7 | 10 | 2.6 | | | very | 1.2*10⁶ *⁾ |
| | moli adhering to it | | | | | | good | |
| 2 | 3. Moli after | 6.9 | 2.8 | 6.5 | 30 | 8.2 | | |
| | carbonation | | | | | | | |
| | 3. Precipitate and | 27.3 | 11.3 | 6.4 | | | acceptable | 2.3*10⁹ *⁾ |
| | moli adhering to it | | | | | | | |
| | 2. Moli (starting | 7.3 | 2.6 | 3.3 | | | | |
| | solution 2 | | | | | | | |
| | for examples 3 to 5) | | | | | | | |
| | 2. Moli after | 13.1 | 4.6 | 5.9 | | | | |
| | concentration | | | | | | | |
| | by factor of 1.8 | | | | | | | |
| 3 | 3. Moli after | 8.2 | 3.4 | 6.1 | 30 | 8.9 | | |
| | carbonation | | | | | | | |
| | 3. Precipitate and | 33.2 | 8.7 | 1.2 | | | very | |
| | moli adhering to it | | | | | | good | |
| 4 | 3. Moli after | 7.6 | 2.6 | 6.2 | 30 | 8.5 | | |
| | carbonation | | | | | | | |
| | 3. Precipitate and | 32.1 | 11.8 | 1 | | | very | |
| | moli adhering to it | | | | | | good | |
| 5 | 3. Moli after | 7.0 | 2.1 | 5.6 | 30 | 8.1 | | |
| | carbonation | | | | | | | |
| | 3. Precipitate and | 29.9 | 12.6 | 5.3 | | | acceptable | |
| | moli adhering to it | | | | | | | |
| | | Concentration in | | | | | | |
| | | % by weight | | | | | | Cake |
| | | | | met- | Carbonation | Carbonation | | resistance |
| Example | Process step | K⁺ | met | met | T in ° C. | pH | Filterability | in m/kg |
| | 2nd Moli (starting | 7.1 | 3.3 | 4.7 | | | | |
| | solution 3 | | | | | | | |
| | for examples 6 to | | | | | | | |
| | 12) | | | | | | | |
| | 2. Moli after | 11.9 | 5.3 | 7.5 | | | | |
| | concentration | | | | | | | |
| | by factor of 1.6 | | | | | | | |
| 6 | 3. Moli after | 8.2 | 5.8 | 8.3 | 30 | 9 | | |
| | carbonation | | | | | | | |
| | 3.Precipitate and | 32.7 | 1.9 | 2.6 | | | very | |
| | moli adhering to it | | | | | | good | |
| 7 | 3. Moli after | 6.4 | 3.6 | 7.6 | 30 | 8.5 | | |
| | carbonation | | | | | | | |
| | 3.Precipitate and | 28.3 | 8.9 | 2.9 | | | very | |
| | moli adhering to it | | | | | | good | |
| 8 | 3. Moli after | 6.2 | 3.1 | 7.3 | 30 | 8.25 | | |
| (comp.) | carbonation | | | | | | | |
| | 3. Precipitate and | 22.7 | 9.3 | 6.8 | | | poor | |
| | moli adhering to it | | | | | | | |
| 9 | 3. Moli after | 9.5 | 5.8 | 8.2 | 55 | 8.97 | | |
| | carbonation | | | | | | | |
| | 3. Precipitate and | 33.1 | 1.8 | 2.6 | | | very | |
| | moli adhering to it | | | | | | good | |
| 10 | 3. Moli after | 8.5 | 5.9 | 8.3 | 55 | 8.53 | | |
| | carbonation | | | | | | | |
| | 3. Precipitate and | 34.0 | 1.6 | 2.3 | | | very | |
| | moli adhering to it | | | | | | good | |
| 11 | 3. Moli after | 8.0 | 4.8 | 8.6 | 50 | 8.47 | | |
| | carbonation | | | | | | | |
| | 3. Precipitate and | 32.4 | 6.2 | 1.3 | | | good | |
| | moli adhering to it | | | | | | | |
| 12 | 3. Moli after | 5.7 | 2.9 | 7.4 | 25 | 8.27 | | |
| (comp.) | carbonation | | | | | | | |
| | 3. Precipitate and | 24.6 | 10.5 | 9.2 | | | very | |
| | moli adhering to it | | | | | | poor | |

Note on table 1:
*⁾ The initially experimentally determined filtration resistance based on filtercake area α_{H} [1/m²] was converted to the filtration resistance α_{M} [m/kg] based on the dry cake mass by division by the density of the dry filtercake ρₛ [kg/m³] according to the formula: α_{M} [ m/kg] = α_{H} [1/m²] / ρₛ [kg/m³] (see ex. 1 and ex. 2) using the following experimentally determined values: α_{H} = 1*10⁹ *1/m² where ρₛ 720 kg/m³ in ex. 1 and α_{H} = 1*10¹⁰ *1/m² where ρₛ 720 kg/m³ in ex. 2.

### Commentary on Table 1

It is apparent from example 1 and 2 in table 1 that the occurrence of met-met in the precipitate can be avoided by increasing the pH to an optimal range. Increasing the temperature in the carbonation step or reducing the concentration factor can also prevent met-met from precipitating and thus improve filterability. However, such a change in these parameters also severely reduces the amount of methionine and potassium in the precipitate. This is the case only to a reduced extent when increasing the pH. Adjustment by pH has therefore surprisingly proven to be a very effective and also simple and cost-effective and thus economically most advantageous solution.

A comparison of examples 3-5 elucidates the effects of the pH on the concentrations measured in the filtrate and the precipitate. The met-met concentration in the concentrated 2nd mother liquor was 5.9% by weight. In example 3 this mother liquor was carbonated up to a pH of 8.9 at 30° C. The met-met concentration in the resulting 3rd mother liquor increased to 6.1% by weight. The reason for this is that the components methionine and KHCO₃ had partially precipitated since the potassium concentration in the 3rd mother liquor fell from 13.1% by weight to 8.2% by weight and the methionine concentration fell from 4.6% to 3.4%. The precipitation of these components brings about an increase in the met-met concentration, provided that met-met does not likewise precipitate. The precipitate was found to contain only 1.2% by weight of met-met. The concentration measurements in the filtrate and cake show that no met-met had precipitated here.

The same effect is apparent in example 4. In the 3rd mother liquor the concentration of methionine and potassium fell further due to the low pH of 8.5 and the met-met concentration increased further to 6.2% by weight.

Only in the carbonation to pH 8.1 in example 5 did the met-met concentration in the filtrate fall to below the starting concentration of 5.9% by weight with a concentration of 5.3% by weight. In addition the met-met concentration in the precipitate is 5.3% by weight here. The filterability of the cake underwent a marked deterioration: from “very good” to “acceptable”.

A comparison of the examples 6, 7 and comparative example 8 again elucidates the effect of the pH. All three examples started from the same starting solution (2nd moli after concentration) and these were carbonated at the same temperature of 30° C. The pH values established were 9.0, 8.5 and 8.2. The solubility and thus the concentration of the value substances potassium and methionine and of the byproduct met-met in the 3rd mother liquor decreases with pH. In comparative example 8 the precipitated met-met proportion in the precipitate of 6.8% by weight was already sufficiently high for the filterability to be poor relative to the very good filterabilities in examples 6 and 7.

A comparison of example 5 with example 8 elucidates the effect of the concentration factor on the met-met concentration of the concentrated 2nd mother liquor which is directly dependent on this factor. In example 8 the precipitate already had “poor” filterability at a pH of 8.25. The reason for this is that the met-met concentration of the starting solution was higher (here 7.5% by weight relative to 5.9% by weight for example 5). The solubility limit of met-met was thus already exceeded at higher pH values and there was accordingly already an appreciable met-met proportion in the precipitate at higher pH values which correspondingly reduced the filterability. The higher the met-met concentration in the starting solution, the higher the pH above which the met-met proportion in the precipitate appreciably increases. The lower the met-met concentration in the starting solution, the lower the pH that results in an appreciable met-met proportion in the precipitate. Below a met-met concentration of the starting solution of 4.5% by weight, the solution (at customary pressure and temperature) can be carbonated to CO₂ saturation (i.e. the lowest achievable pH at the respective pressure) without an appreciable met-met proportion in the precipitate being expected. Since the previously produced mother liquors of the 1st and 2nd carbonation generally still have a content of less than 4.5% by weight of met-met the problem of poor filterability after the carbonation does not occur here. The 2nd mother liquor is enriched in met-met relative to titratable potassium and methionine since these two substances have been removed from the solution by the carbonation and subsequent filtration. In order that in the 3rd carbonation too potassium hydrogencarbonate and/or potassium carbonate and methionine undergo precipitation in the precipitate the 2nd mother liquor must first be concentrated. The concentrated starting solution for the 3rd carbonation (concentrated 2nd mother liquor) therefore achieves higher met-met concentrations relative to the preceding concentrations and so the inventive solution for improving the filterability of the precipitate is brought to bear here in particular.

A comparison of examples 4, 10 and 11 elucidates the effect of temperature. In all three examples the pH is about 8.5. The solubility and thus the concentration of the value substances potassium and methionine in the 3rd mother liquor decreases with temperature.

A comparison of examples 4 and 11 demonstrates the effect of temperature particularly well. In both examples the starting solutions are similarly concentrated and the pH values established in the carbonation are practically identical while the temperature in example 4 is 30° C. and in example 11 is 50° C. However, in example 4 the met-met concentration in the precipitate of 1% by weight is at a similarly low level to that in example 11 of 1.3% by weight. The amount of met-met undergoing precipitation in the precipitate increases with decreasing temperature. Here too, the concentration of the value substances methionine and potassium decrease with temperature.

However, it is apparent from the measured results in table 1 that at a carbonation at pH 9.0 according to example 6 only small amounts of 2.6% by weight met-met were found in the precipitate. The met-met concentration in the 3rd mother liquor is higher than in the concentrated 2nd mother liquor and the met-met concentration in the precipitate is low at 2.6% by weight. This 2.6% by weight may also have been caused by adhering mother liquor.

In the case of carbonation up to pH 8.2 the met-met concentration in the 3rd mother liquor is lower than in the concentrated 2nd mother liquor and in addition the met-met concentration in the precipitate is 6.4% by weight. This indicates that met-met is present in the precipitate.

The cake resistance was measured both for the carbonation at pH 8.7 (1.2*10⁶ m/kg, see example 1) and for the carbonation at pH 8.2 (about 2.3*10⁹ m/kg, see example 2 (comparison)). The cake resistance of the precipitate obtained at pH 8.2 is about 2000 times higher than the resistance of the cake at pH 8.7. Accordingly the filterability of the precipitate at pH 8.7 is very good and thus markedly better than the only acceptable filterability at pH 8.2.

It can be concluded from the examples that the solubilities of the components methionine, potassium (mainly in the form of potassium hydrogen carbonate) and met-met decrease with temperature and pH. The concentration factor in particular ensures a large difference between the concentrations in the concentrated 2nd mother liquor and the 3rd mother liquor and thus ensures a greater mass of the precipitate (=value substance) and a small mass of the 3rd Mother liquor (=byproduct stream).

### DESCRIPTION OF THE FIGURE

The FIGURE shows a scheme for the methionine process according to the invention.

The scheme comprises the following steps, wherein the reference numerals are as defined in table 2 below.

TABLE 2
List of reference numerals for FIG. 1

| Reference | |
|---|---|
| numeral | Process step |
| (1) | Hydrolysis step |
| (2) | 1st carbonation |
| | 1st crystallization (of 1st precipitate) |
| | 1st solid-liquid separation (of 1st precipitate and 1st mother liquor) |
| (3) | 1st concentration (of 1st mother liquor) |
| (4) | Recycling (of 1st portion of the concentrated 1st mother liquor) |
| (5) | 2nd carbonation (of 2nd portion of the concentrated 1st mother |
| | liquor) |
| | 2nd crystallization (of 2nd precipitate) |
| | 2nd solid-liquid separation (of 2nd precipitate and 2nd mother |
| | liquor) |
| (6) | Concentration (of 2nd mother liquor) |
| | 3rd carbonation (of 2nd portion of the concentrated 1st mother |
| | liquor) |
| | 3rd crystallization (of 3rd precipitate) |
| | 3rd solid-liquid separation (of 2nd precipitate and 2nd mother |
| | liquor) |

1. A process for obtaining mixtures comprising methionine and potassium hydrogencarbonate from an aqueous solution or a suspension comprising titratable potassium in the form of potassium hydrogencarbonate and/or potassium carbonate, methionine and 4.5 to 12.0% by weight of methionyl-methionine (met-met), the process comprising:
carbonating the aqueous solution or the suspension with CO₂ at a temperature of 15 to 60° C. to obtain the mixture comprising methionine and potassium hydrogencarbonate as a precipitate; and separating the precipitate from the mixture,
wherein the precipitate comprises 6.5% or less by weight of met-met.

2. The process of claim 1, 1, wherein the aqueous solution or the suspension comprises 6.0 to 18.0% by weight of titratable potassium in the form of potassium hydrogencarbonate and/or potassium carbonate, 2.5 to 8.0% by weight of methionine, and 4.5 to 12.0% by weight of methionyl-methionine.

3. The process of claim 1, wherein the carbonating comprises supplying the aqueous solution or the suspension with CO₂ at a temperature of 15 to 60° C. until a pH of 7.8 to 9.5 is achieved, the pH being measured with a glass electrode at the established temperature,
wherein the aqueous solution or the suspension comprise 6.0 to 18.0% by weight of titratable potassium in the form of potassium hydrogencarbonate and/or potassium carbonate, 2.5 to 8.0% by weight of methionine, and 4.5 to 12.0% by weight of methionyl-methionine.

4. The process of claim 1, wherein the carbonating comprises supplying CO₂ to the aqueous solution or the suspension at a temperature of 25 to 55° C. until a pH of 8.3 to 9.5 is achieved,
wherein the precipitate comprises 0.01% to 5.0% by weight of met-met.

5. The process of claim 1, wherein the carbonating comprises supplying CO₂ to the aqueous solution or the suspension at a temperature of 25 to 55° C. until a pH of 8.4 to 9.5 is achieved,
wherein the precipitate comprises 0.01% to 3.0% by weight of met-met.

6. The process of claim 1, wherein the carbonating comprises supplying CO₂ to the aqueous solution or the suspension until a pH of 8.4 to 9.0 is achieved.

7. The process of claim 1, wherein a pressure during the carbonating is 1 to 6 bara.

8. The process of claim 1, wherein a residence time in the carbonating is 20 to 180 minutes.

9. The process of claim 1, wherein the aqueous solution or the suspension is a mother liquor obtained from an isolation of methionine in a process for producing methionine via alkaline methionine hydantoin saponification.

10. The process of claim 9, wherein the mother liquor is a mother liquor obtained from filtration after repeated carbonation.

11. A mixture comprising methionine and potassium hydrogencarbonate obtained by the process of claim 1.

12. The mixture of claim 11, wherein the mixture further comprises met-met in an amount of 6.5% by weight or less.

13. A method for producing methionine, comprising:
reacting the mixture of claim 11.

14. A process for producing methionine, comprising:
(1) hydrolyzing 5-[2-(methylthio)ethyl]imidazolidin-2,4-dione in the presence of a basic potassium compound to obtain a hydrolysate comprising potassium, methioninate, and potassium methionyl methioninate;
(2) introducing carbon dioxide in a first carbonation into the hydrolyzate to obtain a suspension comprising methionine, methionyl-methionine, and potassium hydrogencarbonate and a methionine precipitate, then separating the suspension into a methionine-containing first precipitate and a methionyl-methionine-containing first mother liquor;
(3) concentrating the first mother liquor obtained in (2) in a first concentration to obtain a concentrated first mother liquor,
(4) recycling a first portion of the concentrated first mother liquor obtained in (3) into (1),
(5) introducing carbon dioxide into a second portion of the concentrated first mother liquor (3) in a second carbonation to obtain a suspension comprising a methionine and a potassium hydrogencarbonate precipitate, then separating the suspension into a second precipitate and a second mother liquor,
(6) concentrating the second mother liquor, then introducing carbon dioxide into the concentrated second mother liquor to obtain a suspension comprising a methionine and a potassium hydrogencarbonate precipitate, then separating the suspension into a third precipitate comprising a mixture of methionine and potassium hydrogencarbonate and a third mother liquor comprising methionyl-methionine,
wherein the third precipitate comprises 6.5% by weight or less of met-met.

15. The process of claim 14, wherein the third precipitate comprises 0.01% to 5.0% by weight of met-met.

16. The process of claim 14, further comprising:
recycling the third precipitate into any of (1) to (4).

17. The process of claim 14, further comprising:
combining the first mother liquor and the third precipitate and subsequently recycling them into (1) or (3).

18. The process of claim 14, further comprising:
disposing of the third mother liquor or supplying it to a further isolation of potassium hydrogencarbonate and/or methionine.
